(19) United States
(12) Patent Application Publication
(10) Pub. No.: US 2026/0011413 A1
(43) Pub. Date: Jan. 8, 2026

(54) SYSTEMS AND METHODS FOR INTERIM CLINICAL TRIAL ANALYSIS

(71) Applicant: Kenvue Brands LLC, Summit, NJ (US)

(72) Inventors: Mark J. Watt, Henley on Thames (GB); Christian Giovanni Scavetta, Oxford (GB); Leticia Busana Galvao Bueno, Ickenham (GB)

(21) Appl. No.: 19/258,115

(22) Filed: Jul. 2, 2025

Related U.S. Application Data
(60) Provisional application No. 63/667,577, filed on Jul. 3, 2024.

(51) Int. Cl. G16H 10/20

(52) U.S. Cl. CPC G16H 10/20

(57) ABSTRACT

Systems and methods for training a clinical trial analysis model for an interim clinical trial analysis and performing an interim clinical trial analysis. The method includes capturing, by a trained clinical trial model analyzer, data associated with an ongoing clinical trial of a novel intervention; performing, by the trained clinical trial model analyzer, an interim analysis of the ongoing clinical trial, wherein performing the interim analysis of the ongoing clinical trial including generating a prediction of safety of the novel intervention, efficacy of the novel intervention, and futility of the novel intervention; comparing, by the trained clinical trial model analyzer, each of the predicted safety of the novel intervention, efficacy of the novel intervention, and futility of the novel intervention to a respective threshold; and based on the comparison, determining, by the trained clinical trial model analyzer, generating a recommendation to terminate the ongoing clinical trial.

### CROSS-REFERENCE TO RELATED APPLICATION

This application claims the benefit of U.S. Provisional Application No. 63/667,577 filed July 3, 2024, the contents of which is incorporated herein by reference in its entirety.

### BACKGROUND

Traditional planning and execution of a clinical trial involves the estimation of potential clinical effects of a novel intervention to determine sample size and power prior to recruitment, as well as a blinded, randomized treatment. The analysis of the clinical trial typically includes an analysis of the results of the clinical trial, such as whether the results indicate the novel intervention was safe and effective. However, clinical trial size and duration is often based on estimates of effect, and when conducted in a blinded fashion may continue past a point at which these results could have been determined, resulting in a waste of time and resources, and lengthening the time it may take to make a treatment available to patients. Attempts to perform an interim analysis to identify such an efficacy point would involve breaking the blind of a study, and potentially compromise the outcome of the clinical trial.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Various implementations of the present disclosure described herein are directed to systems and methods that train a clinical trial analysis model for an interim clinical trial analysis and performing an interim clinical trial analysis. In one implementation, a computer-implemented method is provided. The computer-implemented method includes capturing, by a trained clinical trial model analyzer, data associated with an ongoing clinical trial of a novel intervention; performing, by the trained clinical trial model analyzer, an interim analysis of the ongoing clinical trial, wherein performing the interim analysis of the ongoing clinical trial including generating a prediction of safety of the novel intervention, efficacy of the novel intervention, and futility of the novel intervention; comparing, by the trained clinical trial model analyzer, each of the predicted safety of the novel intervention, efficacy of the novel intervention, and futility of the novel intervention to a respective threshold; and based on the comparison, determining, by the trained clinical trial model analyzer, generating a recommendation to terminate the ongoing clinical trial.

In another implementation, a system is provided. The system includes a memory; a processor coupled to the memory; and an overtrained clinical trial model analyzer implemented on the processor. The processor is configured to capture data associated with an ongoing clinical trial of a novel intervention and generate the overtrained clinical trial model analyzer by overtraining a previously trained clinical trial model analyzer. The overtrained clinical trial model analyzer is configured to perform an interim analysis of the ongoing clinical trial, wherein performing the interim analysis of the ongoing clinical trial including generating a prediction of safety of the novel intervention, efficacy of the novel intervention, and futility of the novel intervention; compare each of the predicted safety of the novel intervention, efficacy of the novel intervention, and futility of the novel intervention to a respective threshold; and based on the comparison, generate a recommendation to terminate the ongoing clinical trial.

In another implementation, one or more non-transitory computer-readable media is provided. The one or more non-transitory computer-readable media stores instructions that, when executed by a processor, cause the processor to execute a clinical trial model trainer implemented on the processor and configured to: receive first data associated with a clinical trial; collect second data from one or more comparative studies based on pre-defined selection criteria, wherein the pre-defined selection criteria is based on parameters derived from the first data associated with the clinical trial; normalize the collected second data; train a plurality of classifiers using the normalized second data as training data; and determine each of the plurality of classifiers is trained to a threshold level of confidence.

### BRIEF DESCRIPTION OF THE DRAWINGS

The present description will be better understood from the following detailed description read in light of the accompanying drawings, wherein:

FIG. 1 illustrates an example system for interim clinical trial analysis according to an example;

FIG. 2 illustrates an example computer-implemented method of performing an interim clinical trial analysis according to an example;

FIG. 3 illustrates an example computer-implemented method of training an artificial intelligence (AI) model for performing interim clinical trial analysis according to an example;

FIG. 4 illustrates an example computer-implemented method of intentional overtraining of the trained AI model for performing interim clinical trial analysis according to an example;

FIG. 5 illustrates an example computer-implemented method of performing an interim clinical trial analysis according to an example;

FIG. 6 illustrates example results of an interim clinical trial analysis according to an example; and

FIG. 7 is a block diagram illustrating an example computing environment suitable for implementing one or more of the various examples disclosed herein.

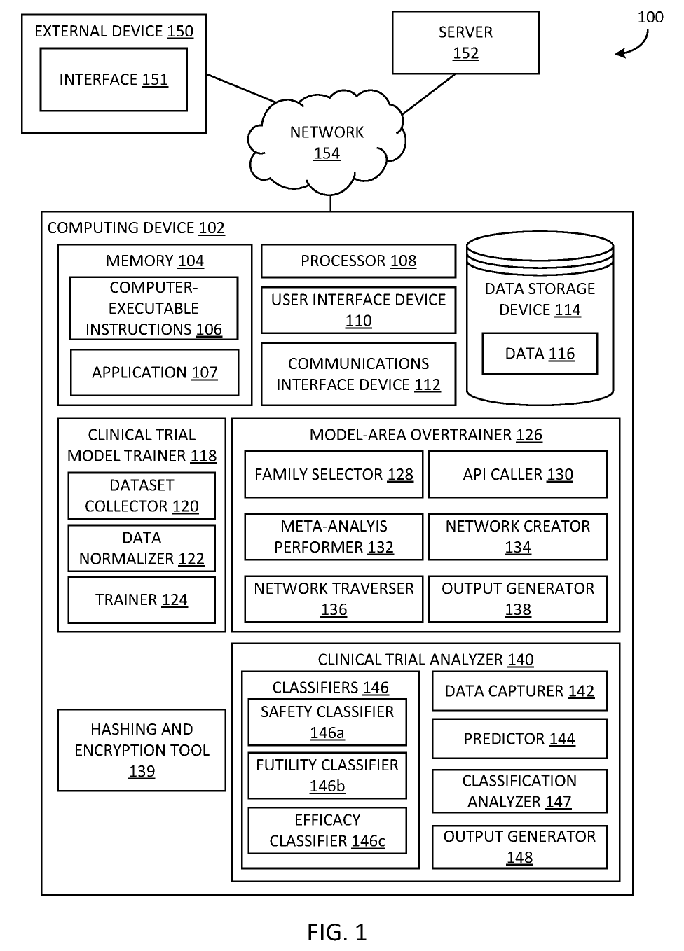

Corresponding reference characters indicate corresponding parts throughout the drawings. In FIGS. 1 to 7, the systems are illustrated as schematic drawings. The drawings may not be to scale.

### DETAILED DESCRIPTION

The various implementations and examples will be described in detail with reference to the accompanying drawings. Wherever possible, the same reference numbers will be used throughout the drawings to refer to the same or like parts. References made throughout this disclosure relating to specific examples and implementations are provided solely for illustrative purposes but, unless indicated to the contrary, are not meant to limit all examples.

As described herein, clinical trials sometimes continue past a point at which these results could have been determined, resulting in a waste of time and resources. Current solutions include performing a planned interim analysis to assess the comparative safety of trial arms for potential termination of the study. However, the true outcome is not known until the end of the study and the blindness is broken. Accordingly, interim analyses fail to effectively assess the comparative safety of trial arms for potential termination of the study, because to effectively assess the intervention would eliminate the blindness, and therefore the reliability, of the trial.

Various examples of the present disclosure recognize and take into account these challenges and provide systems and methods for performing an interim analysis of a clinical trial to monitor, predict, and profile the likely outcomes of a clinical trial in real time, including enabling trial conclusion when endpoints have been reached, while maintaining the blindness of the clinical trial so as to track outcomes of an intervention, namely efficacy and safety of the intervention, in real time. This is performed using a system that includes interposed disaffected artificial intelligence (AI) that enables studies to be monitored to the point of an optimal analysis and outcome at a defined threshold, and advise on discontinuation in real time. This enables early trial cessation where appropriate, saving exposure of patients, reducing study duration, and expenses associated with clinical trials.

The systems and methods for performing an interim analysis of a clinical trial operate in an unconventional manner by performing a combination of training of an AI model that performs the interim analysis as well as identifying a more specific and targeted set of comparative studies that are used to overtrain of the AI model in a specific, niche area on the most similar comparative studies available to an anticipated clinical trial to be analyzed. This provides both a broad view of comparative studies for a class of potential interventions as well as a very targeted understanding of the most similar comparative studies available for a similar subclass of potential interventions based on the additional, targeted overtraining. In addition, the systems and methods described herein further operate in an unconventional manner by performing the interim analysis of the clinical trial while maintaining the blindness of the clinical trial being analyzed by obfuscating the housing of the trained model performing the interim analysis from the actual performance of the clinical trial.

Accordingly, the systems and methods provides a technical solution to the inherently technical problem of performing interim analysis of a clinical trial while maintaining the blindness of the clinical trial by bifurcating the components that perform i) the clinical trial itself, and ii) the interim analysis, while maintaining an ability for each component to effectively perform its aspect of the analysis. In other words, various examples of the present disclosure obfuscate the interim analysis performed at interposed, disaffected location from where the clinical trial is performed, while simultaneously providing a more robust and timely interim analysis than currently available solutions. Accordingly, the technical solutions provided herein enable the tracking of track outcomes of an intervention, namely efficacy and safety, in real time using so that studies may be monitored to the point of an optimal analysis and outcome at a defined threshold and advising on discontinuation in real time, including an identification of an optimal point in the clinical trial at which to conduct the interim analysis. This enables early trial cessation, saving exposure of patients, reducing study duration, and expense of unnecessarily continuing a clinical trial.

As referenced herein, a clinical trial is a systematic research study conducted to evaluate the safety, efficacy, and potential benefits of a novel intervention in humans. These interventions may include, but are not limited to, new drug candidates, new formulations of existing drugs, new dosages or routes of administration, repurposed drugs for alternative therapeutic uses, innovative combinations of treatments, emerging medical devices, supplements, nutritional, psychiatric or psychological therapies or techniques, and so forth. The primary goals of a clinical trial are to determine the intervention's therapeutic value, monitor its side effects, and establish its overall impact on health outcomes. This research is essential for regulatory approval and informed clinical practice.

FIG. 1 illustrates an example system for an interim clinical trial analysis according to an example. The system 100 illustrated in FIG. 1 is provided for illustration only. Other examples of the system 100 can be used without departing from the scope of the present disclosure. In some examples, the system 100 trains an AI model for an interim clinical trial analysis and performs the interim clinical trial analysis while maintaining blindness of the clinical trial according to one or more examples described herein.

The system 100 includes a computing device 102, an external device 150, a server 152, and a network 154. The computing device 102 represents any device executing computer- executable instructions 106 (e.g., as application programs, operating system functionality, or both) to implement the operations and functionality associated with the computing device 102. The computing device 102 in some examples includes a mobile computing device or any other portable device. A mobile computing device includes, for example but without limitation, a mobile telephone, laptop, tablet, computing pad, netbook, gaming device, and/or portable media player. The computing device 102 can also include less-portable devices such as servers, desktop personal computers, kiosks, or tabletop devices. Additionally, the computing device 102 can represent a group of processing units or other computing devices.

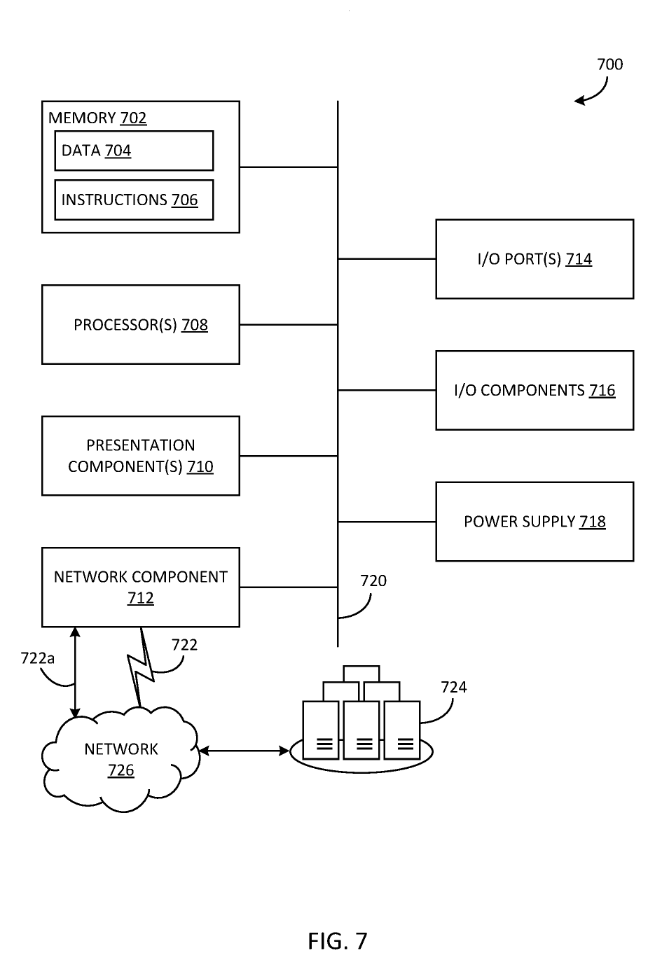

In some examples, the computing device 102 includes at least one processor 108, a memory 104 that includes the computer-executable instructions 106, and a user interface device 110. The processor 108 includes any quantity of processing units and is programmed to execute the computer-executable instructions 106. The computer-executable instructions 106 are performed by the processor 108, performed by multiple processors within the computing device 102, or performed by a processor external to the computing device 102. In some examples, the processor 108 is programmed to execute computer-executable instructions 106 such as those illustrated in the figures described herein, such as FIG. 7. In various examples, the processor 108 is configured to execute computer-executable instructions of one or more of a clinical trial model trainer 118, a model-area overtrainer 126, and a clinical trial analyzer 140.

The memory 104 includes any quantity of media associated with or accessible by the computing device 102. In some examples, the memory 104 is internal to the computing device 102. In other examples, the memory 104 is external to the computing device 102 or both internal and external to the computing device 102. For example, the memory 104 can include both a memory component internal to the computing device 102 and a memory component external to the computing device 102, such as the server 152. The memory 104 stores data, such as one or more applications 107. The applications 107, when executed by the processor 108, operate to perform various functions on the computing device 102. The applications 107 can communicate with counterpart applications or services, such as web services accessible via the network 154. In an example, the applications 107 represent server-side services of an application executing in a cloud, such as a cloud server 152. In some examples, the application 107 is an application for performing an interim clinical trial analysis.

The user interface device 110 includes a graphics card for displaying data to a user and receiving data from the user. The user interface device 110 can also include computer-executable instructions, for example a driver, for operating the graphics card. Further, the user interface device 110 can include a display, for example a touch screen display or natural user interface, and/or computer-executable instructions, for example a driver, for operating the display. The user interface device 110 can also include one or more of the following to provide data to the user or receive data from the user: speakers, a sound card, a camera, a microphone, a vibration motor, one or more accelerometers, a BLUETOOTH® communication module, global positioning system (GPS) hardware, and a photoreceptive light sensor. In a non-limiting example, the user inputs commands or manipulates data by moving the computing device 102 in one or more ways.

The computing device 102 further includes a communications interface device 112. The communications interface device 112 includes one or more of a transceiver, a network interface card, and/or computer-executable instructions, such as a driver, for operating the network interface card. Communication between the computing device 102 and other devices, such as but not limited to the external device 150, can occur using any protocol or mechanism over any wired or wireless connection.

The computing device 102 further includes a data storage device 114 for storing data 116. The data 116 includes, but is not limited to, data associated with a clinical trial for which an interim analysis is to be performed, pre-defined selection criteria for comparative studies to be used for training the clinical trial analyzer, refined selection criteria for overtraining the clinical trial analyzer in a particular area, data associated with the comparative studies, and previous recommendations generated by the clinical trial analyzer for a particular trial.

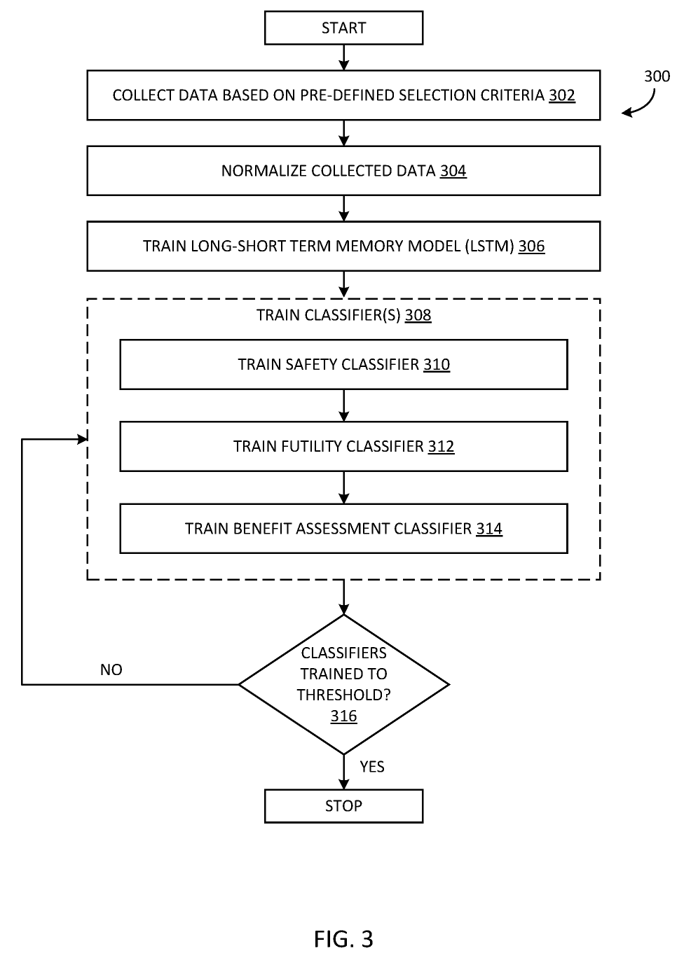

The clinical trial model trainer 118 is an example of a specialized processing unit implemented on the processor 108 that trains an AI model, such as a clinical trial analyzer 140, for analyzing the results of a clinical trial. The clinical trial model trainer 118 includes a dataset collector 120 that collects data from one or more publicly available datasets based on pre-defined selection criteria, a data normalizer 122 that normalizes the collected data, and a trainer 124 that trains a long-short term memory (LSTM) model, for example the clinical trial analyzer 140, to learn the baseline of a clinical trial progression, matches the profile of a study to an outcome based on independent classifiers, and continues the training until each classifier is determined to be trained to a level at or above a determined threshold. Each of the dataset collector 120, the data normalizer 122, and the trainer 124 are additional examples of specialized processing units implemented on the clinical trial model trainer 118. The process of training the clinical trial analyzer 140 via the clinical trial model trainer 118 is described in greater detail below with respect to the computer-implemented method 300 illustrated in FIG. 3.

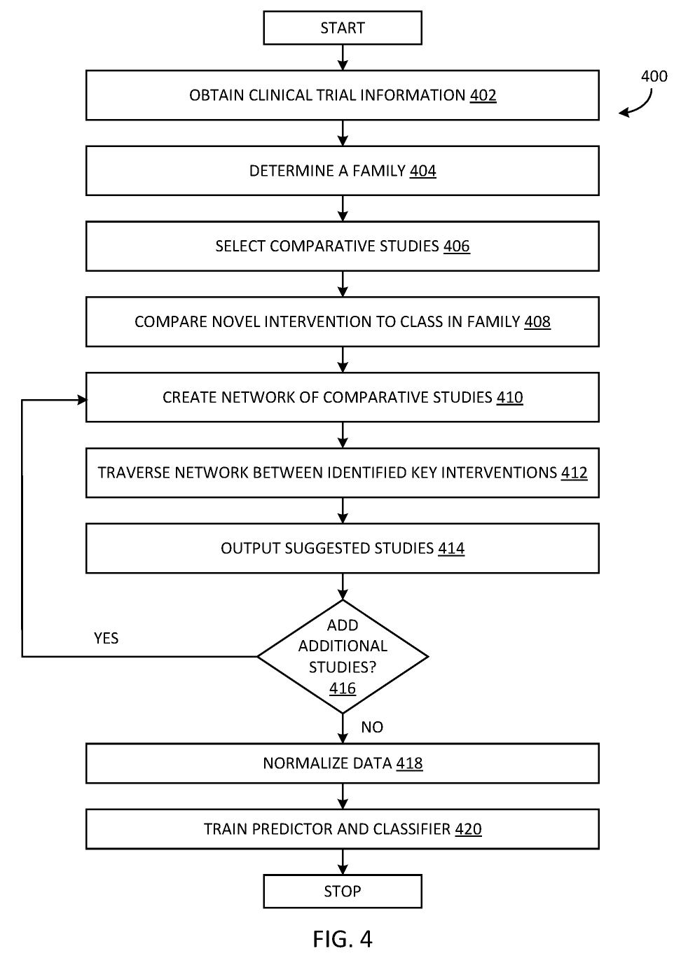

The model-area overtrainer 126 is an example of a specialized processing unit implemented on the processor 108 that overtrains the clinical trial analyzer 140. In some examples, the clinical trial analyzer 140 is trained based on publicly available data associated with the closest clinical trials to a trial to be analyzed by the clinical trial analyzer 140. In other examples, the clinical trial analyzer 140 is trained based on permissive trials, such as previous clinical trial data analyzed by the system or data shared by a client. The model-area overtrainer 126 includes a family selector 128 that identifies the closest available clinical trial or trials to the clinical trial to be analyzed, an application programming interface (API) caller 130 that performs an API call based on parameters of the family of studies identified by the family selector 128, and a meta-analysis performer 132 that compares one or more novel interventions through one or more comparative studies. The model-area overtrainer 126 further includes a network creator 134 that creates, or generates, a network of eligible comparisons for the novel intervention, such as the drug or ingredient candidate, to be analyzed by the clinical trial analyzer 140, a network traverser 136 through which the clinical trial analyzer 140 traverses the generated network to perform the overtraining of the clinical trial analyzer 140, and an output generator 138 that generates an output indicating suggested comparative studies to complete in order to further strengthen the statistical analysis. Each of the family selector 128, the API caller 130, the meta-analysis performer 132, the network creator 134, the network traverser 136, and the output generator 138 are additional examples of specialized processing units implemented on the model-area overtrainer 126. The process of overtraining the clinical trial analyzer 140 via the model-area overtainer 126 is described in greater detail below with respect to the computer-implemented method 400 illustrated in FIG. 4.

The hashing and encryption tool 139 is an example of a specialized processing unit implemented on the processor 108 that sets up the parameters of the clinical trial to be analyzed by the clinical trial analyzer 140. By isolating the concurrent analysis of data blinded from both those performing and those participating in the trial, the blinding of the clinical trial is not compromised, and no interruption of recruitment is needed. Accordingly, the present disclosure operates in an unconventional manner by not introducing bias to the method of the trial and the data that is collected. Furthermore, in an example where the threshold is not met, no action is taken to conclude the clinical trial and the clinical trial may then continue without compromising the blindness of the clinical trial.

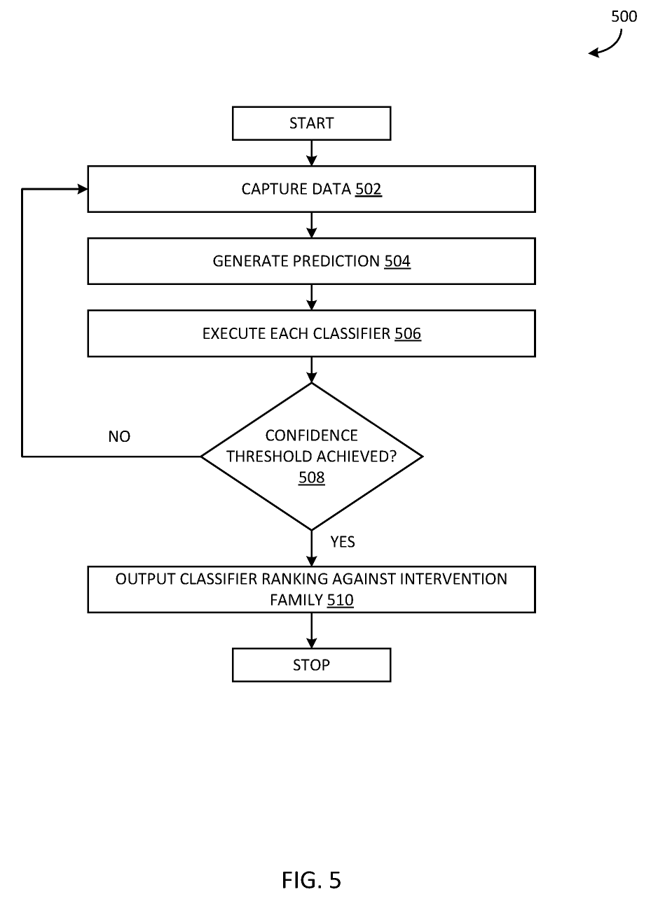

The clinical trial analyzer 140 is an example of a trained AI model, such as the LSTM model described herein, that performs a real-time, interim analysis of a clinical trial to monitor, predict, and profile the likely outcomes of the clinical trial in real time, enabling a determination of when an endpoint of the trial has been reached. In some examples, the clinical trial analyzer 140 is an example of the trained LSTM model trained by clinical trial model trainer 118 and/or the model-area overtrainer 126 prior to implementation on an ongoing clinical trial. The clinical trial analyzer 140 includes a data capturer 142 that captures data from a clinical trial, a predictor 144 that generates a prediction of an outcome of the clinical trial based on the captured data that is available up to that point in time, one or more classifiers 146 that classify the generated prediction as safe or unsafe, effective or noneffective, futile or non-futile, and whether the clinical trial has reached an endpoint, a classification analyzer 147 that analyzes each classification relative to a threshold, and an output generator 148 that generates an output that details the results of the interim analysis of the clinical trial, including a ranking of the novel intervention being tested in the clinical trial against other novel interventions that the clinical trial analyzer 140 has been trained on. Each of the data capturer 142, the predictor 144, the classifiers 146, including a safety classifier 146a, a futility classifier 146b, and an efficacy classifier 146c, the classification analyzer 147, and the output generator 148 are examples of specialized AI models that collectively makes up the clinical trial analyzer 140. The process of performing the interim analysis on a clinical trial via the clinical trial analyzer 140 is described in greater detail with respect to the computer-implemented method 500 illustrated in FIG. 5.

The external device 150 is another example of a computing device, separate from and external of the computing device 102. In some examples, the external device 150 includes a mobile computing device or any other portable device. A mobile computing device includes, for example but without limitation, a mobile telephone, laptop, tablet, computing pad, netbook, gaming device, and/or portable media player. The external device 150 can also include less-portable devices such as servers, desktop personal computers, kiosks, or tabletop devices. Additionally, the external device 150 can represent a group of processing units or other computing devices. The server 152, in some examples, is an example of an external storage device, remote data storage device, a data storage in a remote data center, or a cloud storage. The external device 150 and/or the server 152 communicate with the computing device 102 via the network 154.

In some examples, the external device 150 includes an interface 151. In some examples, the interface is an example of a graphical user interface (GUI) through which a user may review results of the clinical trial, input new data associated with the clinical trial, and review a recommendation associated with termination of the clinical trial that is generated and output by the output generator 148 via the communications interface device 112.

In some examples, the external device 150 is controlled and operated by a separate entity than the computing device 102. By having one interface 151 through which the clinical trial data is input and viewed and a separate computing device 102 that houses the clinical trial analyzer 140 that performs the interim analysis of the clinical trial, the system 100 is able to maintain blindness of the clinical trial while performing the interim analysis. Thus, the present disclosure provides a technical solution to the inherently technical problem of performing interim analysis of a clinical trial while maintaining the blindness of the clinical trial by bifurcating the components that perform i) the clinical trial itself, and ii) the interim analysis, while maintaining a sufficient ability for each system to communicate and effectively perform its aspect of the analysis. The present disclosure minimizes the introduction of bias, retains credibility of results, and maintains trial integrity. By reference to known treatment standards through training, they are potentially more powerful than blinded data safety monitoring boards performing interim safety analyses, as the model itself has no need to be blinded to study arm treatments. In other words, various examples of the present disclosure obfuscate the interim analysis from the parties performing the clinical trial while simultaneously providing a more robust interim analysis than currently available solutions.

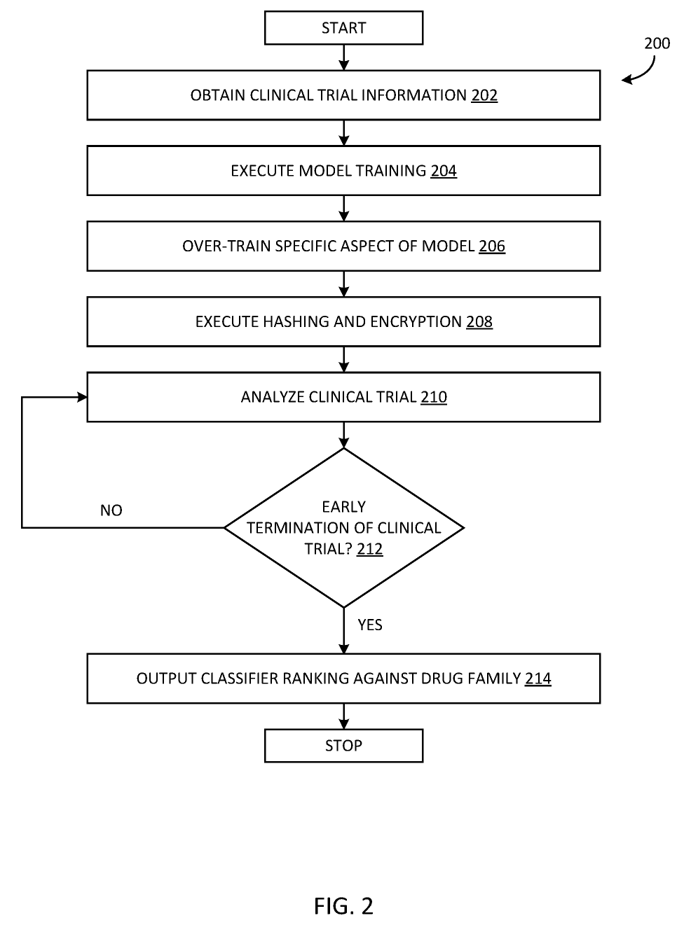

FIG. 2 illustrates an example computer-implemented method of performing an interim clinical trial analysis according to an example. The computer-implemented method 200 is presented for illustration only and should not be construed as limiting. Other examples of the computer-implemented method 200 can be used without departing from the scope of the present disclosure. In some examples, the computer-implemented method 200 is implemented by one or more electronic devices described herein, such as the computing device 102, and in particular the clinical trial model trainer 118, model-area overtrainer 126, hashing and encryption tool 139, and clinical trial analyzer 140 implemented on the computing device 102.

The method 200 begins by the clinical trial trainer 118 obtaining clinical trial information in operation 202. In some examples, the clinical trial information is received via the user interface device 110 on the computing device 102. In other examples, the clinical trial information is received from an external device 150 via the communications interface device 112. The received clinical trial information includes, but is not limited to, data related to the size of the clinical trial, i.e., the number of patients included, patient demographics, an anticipated or planned duration of the clinical trial, any expected adverse events, if applicable, the novel intervention to be tested during the clinical trial, whether the clinical trial includes a control, and scheduling and dosage information of the novel intervention to be tested.

In operation 204, the clinical trial model trainer 118 trains an AI model, such as the clinical trial analyzer 140. For example, the clinical trial model trainer 118 collects data from one or more publicly available datasets based on pre-defined selection criteria, normalizes the collected data, trains the AI model to learn the baseline of a clinical trial progression, matches the profile of a study to an outcome based on independent classifiers, and continues the training until each classifier is determined to be trained to a level at or above a determined threshold. Training of the clinical trial analyze 140 is described in greater detail with respect to the computer-implemented method 300 illustrated in FIG. 3.

In operation 206, the model-area overtrainer 126 over-trains a specific aspect of the AI model, i.e., the clinical trial analyzer 140. The model-area overtrainer 126 identifies the closest available clinical trial or trials to the clinical trial to be analyzed, performs an API call based on parameters of the identified family of studies, compares one or more novel interventions, such as drug or ingredient candidates, through one or more comparative studies, creates a network of eligible comparisons for the novel intervention to be analyzed in the clinical trial, overtrains the clinical trial analyzer 140 via traversing the created network, and generates an output indicating suggested comparative studies to complete in order to further strengthen the statistical analysis. Overtraining the clinical trial analyzer 140 is described in greater detail below with respect to the computer-implemented method 400 illustrated in FIG. 4.

In operation 208, the hashing and encryption tool 139 performs hashing and encryption to set up the parameters of the analysis of the clinical trial. In some examples, the use of appropriate encryption prevents unauthorized users, including investigators or statisticians, from accessing the data of the trial without encryption key. This assists in maintaining the blindness and preventing bias of the clinical trial.

In operation 210, the trained clinical trial analyzer 140 performs an analysis of the identified clinical trial. The clinical trial analyzer 140 captures data from the identified clinical trial, generates a prediction of the results of the clinical trial, and classifies the prediction as safe or unsafe, effective or noneffective, futile or non-futile.

In operation 212, the trained clinical trial analyzer 140 determines whether early termination of the clinical trial is recommended based on the classified prediction. For example, where the prediction indicates that the novel intervention appears to be safe, effective, and non- futile, the trained clinical trial analyzer 140 does not recommend early termination of the clinical trial and returns to operation 208 to continue analysis of the clinical trial. Where the prediction indicates any one of the novel intervention being unsafe, noneffective, or futile, the trained clinical trial analyzer 140 recommends early termination of the clinical trial and proceeds to operation 214.

In operation 214, the trained clinical trial analyzer 140 generates an output detailing the results of the interim analysis of the clinical trial, including a ranking of the novel intervention being tested in the clinical trial against other, historical novel interventions that the clinical trial analyzer 140 has been trained on. For example, the generated output includes details of the recommended early termination of the clinical trial, such as whether the novel intervention is predicted to be unsafe, noneffective, and/or futile. Following operation 214, the computer- implemented method 200 terminates.

FIG. 3 illustrates an example computer-implemented method of training an AI model for performing interim clinical trial analysis according to an example. The computer-implemented method 300 is presented for illustration only and should not be construed as limiting. Other examples of the computer-implemented method 300 can be used without departing from the scope of the present disclosure. In some examples, the computer-implemented method 300 is implemented by one or more electronic devices described herein, such as the clinical trial model trainer 118, as operation 202 of the computer-implemented method 200.

The method 300 begins by the dataset collector 120 collecting data from one or more publicly available datasets from existing clinical trials with which to train the AI model, such as the clinical trial analyzer 140, in operation 302. The dataset collector 120 selects the datasets based on pre-defined selection criteria that set parameters of which datasets are acceptably robust for use in training the AI model. The pre-defined selection criteria includes, but is not limited to, the novel intervention, such as a drug or ingredient candidate or method of treatment, being tested in the study, whether a dataset is sufficiently comparative tested, such as whether the novel intervention tested is compared to one or more similar drugs and a placebo, whether the study includes an acceptable number of participants, such as fifty, one hundred, and so forth, whether the study was peer reviewed, and so forth. In some examples, the collected datasets are obtained using a tailored application programming interface (API) that collects datasets from https://clinicaltrials.gov of clinical trials that match the pre-defined selection criteria. In some examples, the tailored API collects tens of thousands, even a hundred thousand, datasets of individual clinical trials.

In operation 304, the data normalizer 122 normalizes the collected datasets. The data normalizer 122 organizes and/or restructures the collected datasets for consistency between fields and records in the tables that contain the data in order to increase integrity of the collected data and reduce the redundancy of the collected data. This normalization creates a broadly comparable dataset that is used to train the clinical trial analyzer 140. In some examples, the data normalizer 122 employs Z-score normalization to transform each feature to have a mean of zero and a standard deviation of one. This will mitigate the impact of varying scales and units across studies, e.g., the use of 1-10 vs 1-100 in pain studies. In some examples, additional data manipulation may be employed in order to address outliers and other data anomalies.

In operation 306, the trainer 124 trains a LSTM model, such as the clinical trial analyzer 140, to predict a next step in clinical trial sequencing. In some examples, the trainer 124 executes backward propagation of clinical trial data to train the weights of the clinical trial analyzer 140 to match the specific drug candidate. For example, the trainer 124 trains the clinical trial analyzer 140 using the normalized, collected datasets to learn the baseline of clinical trial progression and then match the profile of a study to an outcome based on independent classifiers, such as safety classifier 146a, futility classifier 146b, and efficacy classifier 146c. The clinical trial analyzer 140 is a neural network (NN) that, when trained by the trainer 124, learns the progression of a traditional clinical trial including duration and effectiveness over time. When applied to clinical trials for a particular novel intervention, the trained clinical trial analyzer 140 is able to predict, through Bayesian reasoning, a likely next step in trial sequencing, as well as identifying the point at which a particular clinical trial has reached a point at which it is no longer safe, no longer demonstrating a benefit, and/or has reached a point of futility.

In operation 308, the trainer 124 trains each of three classifiers to match the profile of a clinical trial in the normalized datasets to an outcome. In some examples, the trainer 124 executes backward propagation of the same clinical trial data as is used to train the clinical trial analyzer 140, to train the classifiers on the clinical trial data and respective outcomes. It should be noted that the same clinical trial data is used to train each of the three classifiers as is used to train the clinical trial analyzer 140, which enables the min, max, scaler to be fit to the clinical trial analyzer 140. The profile is an overview of the progression of the relevant studies selected, e.g. time to effect, expected adverse event frequency, and expected adverse event severity. For example, operation 308 includes training the safety classifier 146a in operation 310, training the futility classifier 146b in operation 312, and training the efficacy classifier 146c in operation 314. Although illustrated as occurring in a sequence from operation 310-314, various examples are possible. Operations 310-314 may occur in any sequence, may occur at different times, or may occur simultaneously without departing from the scope of the present disclosure. In operation 310, training the safety classifier 146a including training the safety classifier 146a to classify a clinical trial as safe or unsafe and at which point in the clinical trial such a classification may be made. In operation 312, training the futility classifier 146b including training the futility classifier 14b to classify a clinical trial as futile or non-futile and at which point in the clinical trial such a classification may be made. In operation 314, training the efficacy classifier 146c including training the efficacy classifier 146c to classify a clinical trial as beneficial or non-beneficial and at which point in the clinical trial such a classification may be made. In some examples, the classifiers are trained to perform classification based on the drug, or ingredient, class to be analyzed as the novel intervention. For example, a drug having a known risk of addiction, such as a drug in the opiate class, would have a higher safety threshold than a drug not having a known risk of addiction.

It should be understood that each of the three classifiers 146 are trained separately and, when implemented on a particular clinical trial, perform a separate classification of the clinical trial. In other words, the trained safety classifier 146a makes a safety classification of the clinical trial that is independent of the results of the futility classification made by the futility classifier 146b, and each of these classifications is independent of the results of the benefit assessment, or efficacy, classification made by the efficacy classifier 146c.

In operation 316, the trainer 124 determines whether each of the classifiers are trained to a sufficient threshold. In some examples, the threshold is dynamically generated through random tree classifiers. In other examples, the threshold is manually set and included the clinical trial data prior to the training of the model. In this example, the clinical trial analyzer 140 accounts for this parameter during the training phase. It should be understood that the respective threshold for each classifier is determined independently of the threshold for each other classifier. In other words, the safety classifier 146a is trained to a first threshold, the futility classifier 146b is trained to a second threshold determined independently of the first threshold, and the efficacy classifier 146c is trained to a third threshold determined independently of each of the first threshold and the second threshold. In examples where one or more of the classifiers are determined not to be trained to their respective threshold, the computer-implemented method 300 returns to operation 308 and the trainer 124 continues to train the classifier or classifiers not yet trained to their respective threshold. In examples where each of the classifiers are determined to be trained to their respective thresholds, the computer-implemented method 300 terminates.

FIG. 4 illustrates an example computer-implemented method of intentional overtraining of the trained AI model for performing interim clinical trial analysis according to an example. The computer-implemented method 400 is presented for illustration only and should not be construed as limiting. Other examples of the computer-implemented method 400 can be used without departing from the scope of the present disclosure. In some examples, the computer-implemented method 400 is implemented by one or more electronic devices described herein, such as the model- area overtrainer 126, as operation 204 of the computer-implemented method 200.

The method 400 begins by the model-area overtrainer 126 obtaining clinical trial information regarding an upcoming clinical trial for which the trained clinical trial analyzer 140 is to be implemented in operation 402. In some examples, the clinical trial information is received via the user interface device 110 on the computing device 102. In other examples, the clinical trial information is received from an external device 150 via the communications interface device 112. The received clinical trial information includes, but is not limited to, data related to the size of the clinical trial, i.e., the number of patients included, patient demographics, an anticipated or planned duration of the clinical trial, any expected adverse events, if applicable, the novel intervention to be tested during the clinical trial, any additional parameters of the clinical trial, and scheduling and dosage information of the novel intervention to be tested.

In operation 404, the family selector 128 determines a family for the particular novel intervention based on the received clinical trial information. For example, where the novel intervention is a drug candidate, the family includes a drug family for the drug candidate based on the received clinical trial information. In particular, the family selector 128 determines the drug family primarily based on the drug or ingredient data and secondarily on the scheduling and dosing information. The drug family, also referred to herein as a drug class, is a group of drugs, or ingredients, having similar chemical structures and/or mechanisms of actions that treat similar conditions. In some examples, drugs in a drug family may also have similar side effects. Examples of drug families may be broad or more narrowed and targeted. Broad examples of a drug family include, but are not limited to, examples such as antibiotics, antidepressants, opioids, beta blockers, blood thinners, and so forth. Examples of more narrowed and specific drug families may include, but are not limited to, penicillin-based families within the broader family of antibiotics, selective serotonin reuptake inhibitors (SSRIs) within the broader family of antidepressants, and so forth.

In operation 406, the API caller 130 performs a tailored API call to select one or more datasets, associated with comparative studies, to be used to overtrain the clinical trial analyzer 140. In some examples, the tailored API call performed in operation 406 is similar to the API call performed in operation 302, but is performed with more narrow, detailed parameters than the API call in operation 302. In some examples, these more narrow, detailed parameters are referred as refined selection parameters. For example, the pre-defined selection criteria for a particular drug in the antibiotic class in operation 302 may include drugs in the same antibiotic class having a threshold of fifty subjects, whereas the pre-defined selection criteria in operation 406 include drugs in the same narrow antibiotic class, such as penicillin, that match the received information in operation 402, and a study size between 80 and 100 subjects, corresponding to the received information in operation 402. Thus, the API call in operation 406 selects specific datasets that most closely resemble the clinical trial to be performed such that the clinical trial analyzer 140 may be overtrained in the specific type of clinical trial to be performed.

In operation 408, the meta-analysis performer 132 analyzes the novel intervention through comparative studies. The meta-analysis performers 132 creates a reference rating for the particular novel intervention that compares the novel intervention to other interventions within its intervention class, such as comparing a drug candidate to other drugs within its drug class. The reference rating is a measure of efficacy of the novel intervention relative to the safety rate of the novel intervention and the comparison of the efficacy and safety ratings to other interventions in the class. The analysis of the novel intervention through comparative studies enables additional comparisons that may not have been made directly through any one study. For example, where a first study compares drug A and drug B, and a second study compares drug B and drug C, the meta-analysis performer 132 compares the first study and the second study to perform an additional comparison of drug A to drug C. This results in a more comprehensive view of each intervention and each study, enabling the clinical trial analyzer 140 to ultimately be trained on a more robust dataset than if compared using more traditional training methods that fail to include overtraining.

In operation 410, the network creator 134 creates a network of eligible comparisons for the novel intervention to be analyzed by the clinical trial analyzer 140. In some examples, creating the network of eligible comparisons includes creating an order of nodes associated with the selected comparative studies, based at least in part on the created reference rating for the particular novel intervention, through which the clinical trial analyzer 140 is to traverse in order to be overtrained using the comparative studies. In some examples, the created network includes a suggestion of additional comparative studies or trials to be completed next in order to strengthen a comparison between the novel intervention and another novel intervention.

In operation 412, the network traverser 136 controls the clinical trial analyzer 140 to traverse the created network of nodes between identified key comparison interventions. By traversing the created network of nodes, the clinical trial analyzer 140 determines the strongest comparisons for the novel intervention as well as an anticipated trajectory of the clinical trial based on the comparative studies. For example, by traversing the created network of nodes the clinical trial analyzer 140 learns an anticipated duration of the clinical trial to be completed, information associated with timing where safety, efficacy, and futility are anticipated to be learned, if, when, and to what extend adverse events may be anticipated, and so forth.

In operation 414, the output generator 138 generates an output indicating suggested comparative studies to complete in order to be used to strengthen the statistical analysis. For example, the inclusion of the additionally suggested comparative studies provide an opportunity for additional comparison against another intervention drug and/or to confirm a similar comparison to another intervention. In other words, the identification and inclusion of additional studies provides opportunities for additional comparison to a particular intervention that, if completed, either confirm results of a previous study or avoid a favorable comparison that has only minimal support.

In operation 416, the network creator 134 determines whether or not to include the additionally suggested studies to the created network and, if so, where in the network such studies are to be placed in the created network. In some examples, studies are dynamically added through relation. For example, if a pathway is selected, additional studies that are related to the current node are assessed. Where the network creator 134 determines to include the additionally suggested studies to the created network, the network creator 134 returns to operation 410 and updates the created network with the additionally suggested studies. Where the network creator 134 determines not to include the additionally suggested studies to the created network, the computer-implemented method 400 proceeds to operation 418.

In operation 418, the data normalizer 122 normalizes the collected datasets. As described herein, the data normalizer 122 organizes and/or restructures the collected datasets for consistency between fields and records in the tables that contain the data in order to increase integrity of the collected data and reduce the redundancy of the collected data. This normalization creates a broadly comparable dataset that is used to train the clinical trial analyzer 140. In some examples, the data normalizer 122 employs Z-score normalization to transform each feature to have a mean of zero and a standard deviation of one. This will mitigate the impact of varying scales and units across studies, e.g., the use of 1-10 vs 1-100 in pain studies. In some examples, additional data manipulation may be employed in order to address outliers and other data anomalies.

In operation 420, the trainer 124 trains the clinical trial analyzer 140 and the classifiers, e.g., the safety classifier 146a, futility classifier 146b, and efficacy classifier 146c. For example, the trainer 124 trains the clinical trial analyzer 140 by executing backward propagation of clinical trial data to train the weights of the clinical trial analyzer 140 to match the specific drug candidate, and trains each of three classifiers to match the profile of a clinical trial in the normalized datasets to an outcome by executing backward propagation of the clinical trial data to train the classifiers on the clinical trial data and respective outcomes.

FIG. 5 illustrates an example computer-implemented method of performing an interim clinical trial analysis according to an example. The computer-implemented method 500 is presented for illustration only and should not be construed as limiting. Other examples of the computer-implemented method 500 can be used without departing from the scope of the present disclosure. In some examples, the computer-implemented method 500 is implemented by one or more electronic devices described herein, such as the clinical trial analyzer 140, as operations 208 and 210 of the computer-implemented method 200.

The method 500 begins by the data capturer 142 capturing data of an ongoing clinical trial in operation 502. For example, the clinical trial data includes, but is not limited to, the novel intervention being tested during the clinical trial, data related to the size of the clinical trial, i.e., the number of patients included, patient demographics, a duration of the clinical trial, noted adverse events, if applicable, and scheduling and dosage information of the novel intervention being tested. In some examples, the data is received in response to a request for clinical trial data from the data capturer 142. In other examples, the data is automatically transmitted to the data capturer 142 from the external device 150 via the communications interface device 112 at regular intervals, such as once a day, once a week, and so forth.

In operation 504, the predictor 144 analyzes the captured data regarding the clinical trial and generates a prediction regarding the novel intervention being tested by the clinical trial based on the analysis. The generated prediction includes a prediction of an outcome of the clinical trial based on the captured data that is available up to that point in time. The prediction is generated by a comparison of the captured clinical trial data at a certain point in the clinical trial to comparative studies determined to be the most similar to the current clinical trial. Using the same example as described earlier with reference to FIG. 4, where the clinical trial studies penicillin as the novel intervention over a period of time, comparative studies will include other clinical trials studying penicillin in reasonably similar doses as the novel intervention for a similar use case over a similar period of time. Where the comparative studies determine whether penicillin was safe, effective, and/or futile for a certain use case over a certain period of time, the predictor 144 analyzes the comparative studies at the same point in time as the current clinical trial and compares the current clinical trial data to predict the outcome of the current clinical trial.

In operation 506, each of the classifiers 146 perform an independent, interim classification of the novel intervention being tested in the clinical trial. For example, the safety classifier 146a makes an interim classification of whether the novel intervention is safe or unsafe, the futility classifier 146b makes an interim classification of whether the novel intervention is futile or non- futile, and the efficacy classifier 146c makes an interim classification of whether the novel intervention is effective or non-effective. As described herein, each classification is independent of each other classification.

In operation 508, the classification analyzer 147 analyzes each interim classification of the novel intervention relative to a respective threshold. For example, the classification analyzer 147 compares the safety classification to the first threshold relating to a required safety level of the novel intervention, the futility classification to the second threshold relating to a required futility level of the novel intervention, and the efficacy classification to the third threshold relating to a required efficacy of the novel intervention. Where the novel intervention is classified as any one of unsafe, ineffective, or futile to a respective threshold level indicating a sufficient level of confidence, or where the novel intervention is classified as each of safe, effective, and non-futile to a respective threshold level indicating a sufficient level of confidence, the classification analyzer 147 makes an interim determination that the clinical trial has reached a point at which continuing the trial will yield further results and proceeds to operation 510. Where the classification analyzer 147 fails to make an interim determination that the novel intervention is any one of unsafe, ineffective, or futile to the threshold level of confidence or each of safe, effective, and non-futile to a respective threshold level, i.e., that continuing the clinical trial will yield further results, the computer-implemented method 500 returns to operation 502.

In operation 510, the output generator 148 generates an output detailing the results of the interim analysis of the clinical trial, including a ranking of the novel intervention being tested in the clinical trial against other intervention that the clinical trial analyzer 140 has been trained on. The generated output includes a recommendation that the clinical trial has reached an endpoint due to the confidence level of the results of the clinical trial and an analysis of why, relative to other comparative studies, the clinical trial has reached its endpoint. Following operation 510, the computer-implemented method 500 terminates.

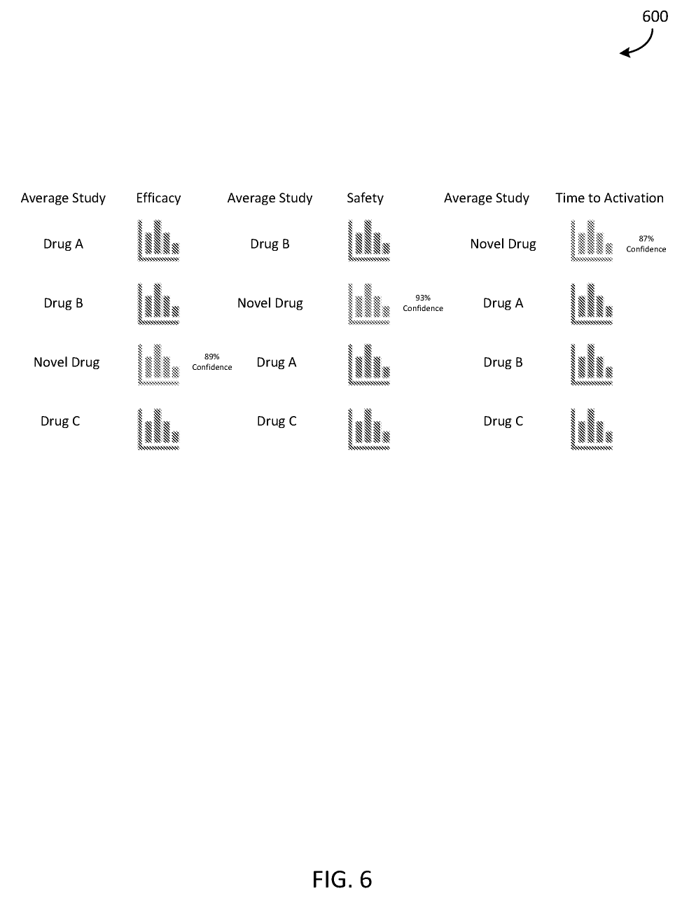

FIG. 6 illustrates example results of an interim clinical trial analysis according to an example. The example results of the interim clinical trial analysis illustrated in FIG. 6 are for illustration only and should not be construed as limiting. Various examples are possible without departing from the scope of the present disclosure.

The example results 600 illustrate efficacy, safety, and time to activation levels for Drug A, Drug B, Drug B, and a Novel Drug. The Novel Drug is an example of a novel intervention currently undergoing a clinical trial. Each of Drugs A, B, and C are examples of drugs that were the subject of comparative studies on which the clinical trial analyzer 140 was trained, for example by the clinical trial model trainer 118 and/or the model-area overtrainer 126. As shown in the example results, the Novel Drug ranks above Drug C for efficacy, above Drugs A and C for safety, and above each of Drugs A, B, and C for time to activation. In some examples, the examples results 600 are included in the generated recommendation that is generated and output by the output generator 148 based on the results of the interim analysis of the clinical trial.

### Example Operating Environment

FIG. 7 is a block diagram of an example computing device 700 for implementing aspects disclosed herein and is designated generally as computing device 700. Computing device 700 is an example of a suitable computing environment and is not intended to suggest any limitation as to the scope of use or functionality of the examples disclosed herein. Neither should computing device 700 be interpreted as having any dependency or requirement relating to any one or combination of components/modules illustrated. The examples disclosed herein may be described in the general context of computer code or machine-useable instructions, including computer- executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples may be practiced in a variety of system configurations, including personal computers, laptops, smart phones, mobile tablets, hand-held devices, consumer electronics, specialty computing devices, etc. The disclosed examples may also be practiced in distributed computing environments when tasks are performed by remote-processing devices that are linked through a communications network.

Computing device 700 includes a bus 720 that directly or indirectly couples the following devices: computer-storage memory 702, one or more processors 708, one or more presentation components 710, I/O ports 714, I/O components 716, a power supply 718, and a network component 712. While computing device 700 is depicted as a seemingly single device, multiple computing devices 700 may work together and share the depicted device resources. For example, memory 702 may be distributed across multiple devices, and processor(s) 708 may be housed with different devices.

Bus 720 represents what may be one or more busses (such as an address bus, data bus, or a combination thereof). Although the various blocks of FIG. 7 are shown with lines for the sake of clarity, delineating various components may be accomplished with alternative representations. For example, a presentation component such as a display device is an I/O component in some examples, and some examples of processors have their own memory. Distinction is not made between such categories as "workstation," "server," "laptop," "hand-held device," etc., as all are contemplated within the scope of FIG. 7 and the references herein to a "computing device." Memory 702 may take the form of the computer-storage media references below and operatively provide storage of computer-readable instructions, data structures, program modules and other data for computing device 700. In some examples, memory 702 stores one or more of an operating system, a universal application platform, or other program modules and program data. Memory 702 is thus able to store and access data 704 and instructions 706 that are executable by processor 708 and configured to carry out the various operations disclosed herein.

In some examples, memory 702 includes computer-storage media in the form of volatile and/or nonvolatile memory, removable or non-removable memory, data disks in virtual environments, or a combination thereof. Memory 702 may include any quantity of memory associated with or accessible by computing device 700. Memory 702 may be internal to computing device 700 (as shown in FIG. 7), external to computing device 700, or both. Examples of memory 702 include, without limitation, random access memory (RAM); read only memory (ROM); electronically erasable programmable read only memory (EEPROM); flash memory or other memory technologies; CD-ROM, digital versatile disks (DVDs) or other optical or holographic media; magnetic cassettes, magnetic tape, magnetic disk storage or other magnetic storage devices; memory wired into an analog computing device; or any other medium for encoding desired information and for access by computing device 700. Additionally, or alternatively, memory 702 may be distributed across multiple computing devices 700, for example, in a virtualized environment in which instruction processing is carried out on multiple computing devices 700. For the purposes of this disclosure, "computer storage media," "computer-storage memory," "memory," and "memory devices" are synonymous terms for computer-storage memory 702, and none of these terms include carrier waves or propagating signaling.

Processor(s) 708 may include any quantity of processing units that read data from various entities, such as memory 702 or I/O components 716 and may include CPUs and/or GPUs. Specifically, processor(s) 708 are programmed to execute computer-executable instructions for implementing aspects of the disclosure. The instructions may be performed by the processor, by multiple processors within computing device 700, or by a processor external to client computing device 700. In some examples, processor(s) 708 are programmed to execute instructions such as those illustrated in the in the accompanying drawings. Moreover, in some examples, processor(s) 708 represent an implementation of analog techniques to perform the operations described herein. For example, the operations may be performed by an analog client computing device 700 and/or a digital client computing device 700. Presentation component(s) 710 present data indications to a user or other device. Exemplary presentation components include a display device, speaker, printing component, vibrating component, etc. One skilled in the art will understand and appreciate that computer data may be presented in a number of ways, such as visually in a graphical user interface (GUI), audibly through speakers, wirelessly between computing devices 700, across a wired connection, or in other ways. I/O ports 714 allow computing device 700 to be logically coupled to other devices including I/O components 716, some of which may be built in. Example I/O components 716 include, for example but without limitation, a microphone, joystick, game pad, satellite dish, scanner, printer, wireless device, etc.

Computing device 700 may operate in a networked environment via network component 712 using logical connections to one or more remote computers. In some examples, network component 712 includes a network interface card and/or computer-executable instructions (e.g., a driver) for operating the network interface card. Communication between computing device 700 and other devices may occur using any protocol or mechanism over any wired or wireless connection. In some examples, network component 712 is operable to communicate data over public, private, or hybrid (public and private) using a transfer protocol, between devices wirelessly using short range communication technologies (e.g., near-field communication (NFC), Bluetooth^{TM} branded communications, or the like), or a combination thereof. Network component 712 communicates over wireless communication link 722 and/or a wired communication link 722a to a cloud resource 724 across network 726. Various different examples of communication links 722 and 722a include a wireless connection, a wired connection, and/or a dedicated link, and in some examples, at least a portion is routed through the internet.

Although described in connection with an example computing device 700, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable and non-removable memory implemented in any method or technology for storage of information such as computer readable instructions, data structures, program modules, or the like. Computer storage media are tangible and mutually exclusive to communication media. Computer storage media are implemented in hardware and are non-transitory, i.e., exclude carrier waves and propagated signals. Computer storage media for purposes of this disclosure are not signals per se. Exemplary computer storage media include hard disks, flash drives, solid-state memory, phase change random-access memory (PRAM), static random-access memory (SRAM), dynamic random-access memory (DRAM), other types of random-access memory (RAM), read- only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

In some examples, a computer-implemented method includes capturing, by a trained clinical trial model analyzer, data associated with an ongoing clinical trial of a novel intervention; performing, by the trained clinical trial model analyzer, an interim analysis of the ongoing clinical trial, wherein performing the interim analysis of the ongoing clinical trial including generating a prediction of safety of the novel intervention, efficacy of the novel intervention, and futility of the novel intervention; comparing, by the trained clinical trial model analyzer, each of the predicted safety of the novel intervention, efficacy of the novel intervention, and futility of the novel intervention to a respective threshold; and based on the comparison, determining, by the trained clinical trial model analyzer, generating a recommendation to terminate the ongoing clinical trial.

In some examples, a system includes a memory; and a processor coupled to the memory, and an overtrained clinical trial model analyzer implemented on the processor. The processor is configured to capture data associated with an ongoing clinical trial of a novel intervention and generate the overtrained clinical trial model analyzer by overtraining a previously trained clinical trial model analyzer. The overtrained clinical trial model analyzer is configured to perform an interim analysis of the ongoing clinical trial, wherein performing the interim analysis of the ongoing clinical trial including generating a prediction of safety of the novel intervention, efficacy of the novel intervention, and futility of the novel intervention; compare each of the predicted safety of the novel intervention, efficacy of the novel intervention, and futility of the novel intervention to a respective threshold; and based on the comparison, generate a recommendation to terminate the ongoing clinical trial.

In some examples, one or more non-transitory computer-readable media stores instructions that, when executed by a processor, cause the processor to execute a clinical trial model trainer implemented on the processor and configured to: receive first data associated with a clinical trial; collect second data from one or more comparative studies based on pre-defined selection criteria, wherein the pre-defined selection criteria is based on parameters derived from the first data associated with the clinical trial; normalize the collected second data; train a plurality of classifiers using the normalized second data as training data; and determine each of the plurality of classifiers is trained to a threshold level of confidence.

Further examples for are described herein.

Various examples further include one or more of the following:

receiving the captured data from an external device;

transmitting, to the external device, the generated recommendation;

wherein comparing each of the predicted safety of the novel intervention, efficacy of the novel intervention, and futility of the novel intervention to the respective threshold further comprises: comparing the predicted safety of the novel intervention to a first threshold, wherein the first threshold is a safety threshold; comparing the predicted efficacy of the novel intervention to a second threshold, wherein the second threshold is an efficacy threshold, and wherein the second threshold is independent of the first threshold; and comparing the predicted futility of the novel intervention to a third threshold, wherein the third threshold is a futility threshold, and wherein the third threshold is independent of each of the first threshold and the second threshold;

performing hashing and encryption of the data associated with the ongoing clinical trial, wherein capturing the data associated with the ongoing clinical trial includes capturing the hashed and encrypted data;

wherein performing the interim analysis of the ongoing clinical trial further comprises: utilizing the captured data to further train the trained clinical trial model analyzer on a baseline progression of the ongoing clinical trial; matching a profile of the ongoing clinical trial to an outcome based on at least one independent classifiers; and training each of the at least independent classifiers to a level at or above a predetermined threshold;

wherein performing the interim analysis of the ongoing clinical trial further comprises: generating an overtrained clinical trial model analyzer by overtraining an aspect of the trained clinical trial model analyzer using one or more eligible comparisons for the novel intervention;

comparing, by the overtrained clinical trial model analyzer, each of the predicted safety of the novel intervention, efficacy of the novel intervention, and futility of the novel intervention to a respective threshold;

wherein the generated recommendation includes a ranking of a measure of each of the predicted safety of the novel intervention, efficacy of the novel intervention, and futility of the novel intervention against other historical novel interventions;

select a drug family based on the received first data; refine the pre-defined selection criteria to a refined selection criteria, the refined selection criteria narrower than the pre-defined selection criteria; collect third data from additional comparative studies based on the refined selection criteria; and overtrain the plurality of classifiers based on the collected third data from additional comparative studies;

generate a reference rating the novel intervention by analyzing the novel intervention in relation to the collected third data from the additional comparative studies, wherein the reference rating is a measure of efficacy of the novel intervention relative to a safety rate of the novel intervention and a comparison of the efficacy and safety ratings to other interventions in the selected drug family;

generate a network of nodes, wherein each node represents an eligible comparisons for the novel intervention; and determine a strongest comparison for the novel intervention by traversing the created network of nodes between identified key comparison interventions; and

wherein the network of nodes is generated based at least in part on the generated reference rating.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles "a," "an," "the," and "said" are intended to mean that there are one or more of the elements. The terms "comprising," "including," and "having" are intended to be inclusive and mean that there may be additional elements other than the listed elements. The term "exemplary" is intended to mean "an example of." The phrase "one or more of the following: A, B, and C" means "at least one of A and/or at least one of B and/or at least one of C."

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A computer-implemented method, comprising:
capturing, by a trained clinical trial model analyzer, data associated with an ongoing clinical trial of a novel intervention;
performing, by the trained clinical trial model analyzer, an interim analysis of the ongoing clinical trial, wherein performing the interim analysis of the ongoing clinical trial including generating a prediction of safety of the novel intervention, efficacy of the novel intervention, and futility of the novel intervention;
comparing, by the trained clinical trial model analyzer, each of the predicted safety of the novel intervention, efficacy of the novel intervention, and futility of the novel intervention to a respective threshold; and
based on the comparison, generating, by the trained clinical trial model analyzer, a recommendation to terminate the ongoing clinical trial.

2. The computer-implemented method of claim 1, further comprising:
receiving the captured data from an external device; and
transmitting, to the external device, the generated recommendation.

3. The computer-implemented method of claim 1, wherein comparing each of the predicted safety of the novel intervention, efficacy of the novel intervention, and futility of the novel intervention to the respective threshold further comprises:
comparing the predicted safety of the novel intervention to a first threshold, wherein the first threshold is a safety threshold;
comparing the predicted efficacy of the novel intervention to a second threshold, wherein the second threshold is an efficacy threshold, and wherein the second threshold is independent of the first threshold; and
comparing the predicted futility of the novel intervention to a third threshold, wherein the third threshold is a futility threshold, and wherein the third threshold is independent of each of the first threshold and the second threshold.

4. The computer-implemented method of claim 1, further comprising:
performing hashing and encryption of the data associated with the ongoing clinical trial, wherein capturing the data associated with the ongoing clinical trial includes capturing the hashed and encrypted data.

5. The computer-implemented method of claim 1, wherein performing the interim analysis of the ongoing clinical trial further comprises:
utilizing the captured data to further train the trained clinical trial model analyzer on a baseline progression of the ongoing clinical trial;
matching a profile of the ongoing clinical trial to an outcome based on at least one independent classifiers; and
training each of the at least independent classifiers to a level at or above a predetermined threshold.

6. The computer-implemented method of claim 5, wherein performing the interim analysis of the ongoing clinical trial further comprises:
generating an overtrained clinical trial model analyzer by overtraining an aspect of the trained clinical trial model analyzer using one or more eligible comparisons for the novel intervention.

7. The computer-implemented method of claim 6, further comprising:
comparing, by the overtrained clinical trial model analyzer, each of the predicted safety of the novel intervention, efficacy of the novel intervention, and futility of the novel intervention to a respective threshold.

8. The computer-implemented method of claim 1, wherein the generated recommendation includes a ranking of a measure of each of the predicted safety of the novel intervention, efficacy of the novel intervention, and futility of the novel intervention against other historical novel interventions.

9. A system, comprising:
a memory;
a processor coupled to the memory and configured to: capture data associated with an ongoing clinical trial of a novel intervention, and generate an overtrained clinical trial model analyzer by overtraining a previously trained clinical trial model analyzer; and the overtrained clinical trial model analyzer implemented on the processor and configured to: perform an interim analysis of the ongoing clinical trial, wherein performing the interim analysis of the ongoing clinical trial including generating a prediction of safety of the novel intervention, efficacy of the novel intervention, and futility of the novel intervention; compare each of the predicted safety of the novel intervention, efficacy of the novel intervention, and futility of the novel intervention to a respective threshold; and based on the comparison, generate a recommendation to terminate the ongoing clinical trial.

10. The system of claim 9, wherein the processor is further configured to control a transceiver to:
receive the captured data from an external device; and
transmit, to the external device, the generated recommendation.

11. The system of claim 9, wherein, to compare each of the predicted safety of the novel intervention, efficacy of the novel intervention, and futility of the novel intervention to the respective threshold, the overtrained clinical trial model analyzer is further configured to:
compare the predicted safety of the novel intervention to a first threshold, wherein the first threshold is a safety threshold;
compare the predicted efficacy of the novel intervention to a second threshold, wherein the second threshold is an efficacy threshold, and wherein the second threshold is independent of the first threshold; and
compare the predicted futility of the novel intervention to a third threshold, wherein the third threshold is a futility threshold, and wherein the third threshold is independent of each of the first threshold and the second threshold.

12. The system of claim 9, wherein the processor is further configured to:
perform hashing and encryption of the data associated with the ongoing clinical trial,
wherein capturing the data associated with the ongoing clinical trial includes capturing the hashed and encrypted data.

13. The system of claim 9, wherein, to perform the interim analysis of the ongoing clinical trial, the overtrained clinical trial model analyzer is further configured to:
utilize the captured data to further train the trained clinical trial model analyzer on a baseline progression of the ongoing clinical trial;
match a profile of the ongoing clinical trial to an outcome based on at least one independent classifiers; and
train each of the at least independent classifiers to a level at or above a predetermined threshold.

14. The system of claim 13, wherein, to generate the overtrained clinical trial model analyzer, the processor is further configured to:
overtrain an aspect of the trained clinical trial model analyzer using one or more eligible comparisons for the novel intervention; and
compare each of the predicted safety of the novel intervention, efficacy of the novel intervention, and futility of the novel intervention to a respective threshold.

15. The system of claim 9, wherein the generated recommendation includes a ranking of a measure of each of the predicted safety of the novel intervention, efficacy of the novel intervention, and futility of the novel intervention against other historical novel interventions.

16. One or more non-transitory computer-readable media storing instructions that, when executed by a processor, cause the processor to execute a clinical trial model trainer implemented on the processor and configured to:
receive first data associated with a clinical trial of a novel intervention;
collect second data from one or more comparative studies based on pre-defined selection criteria, wherein the pre-defined selection criteria is based on parameters derived from the first data associated with the clinical trial; normalize the collected second data; train a plurality of classifiers using the normalized second data as training data; and determine each of the plurality of classifiers is trained to a threshold level of confidence.

17. The one or more non-transitory computer-readable media of claim 16, further storing instructions that, when executed by the clinical trial model trainer, cause the clinical trial model trainer to:
select a drug family based on the received first data;
refine the pre-defined selection criteria to a refined selection criteria, the refined selection criteria narrower than the pre-defined selection criteria;
collect third data from additional comparative studies based on the refined selection criteria; and
overtrain the plurality of classifiers based on the collected third data from additional comparative studies.

18. The one or more non-transitory computer-readable media of claim 17, further storing instructions that, when executed by the clinical trial model trainer, cause the clinical trial model trainer to:
generate a reference rating the novel intervention by analyzing the novel intervention in relation to the collected third data from the additional comparative studies, wherein the reference rating is a measure of efficacy of the novel intervention relative to a safety rate of the novel intervention and a comparison of the efficacy and safety ratings to other interventions in the selected drug family.

19. The one or more non-transitory computer-readable media of claim 18, further storing instructions that, when executed by the clinical trial model trainer, cause the clinical trial model trainer to:
generate a network of nodes, wherein each node represents an eligible comparisons for the novel intervention; and determine a strongest comparison for the novel intervention by traversing the generated network of nodes between identified key comparison interventions.

20. The one or more non-transitory computer-readable media of claim 19, wherein the network of nodes is generated based at least in part on the generated reference rating.
